(19) United States
(12) Patent Application Publication
(10) Pub. No.: US 2019/0170135 A1
(43) Pub. Date: Jun. 6, 2019

(54) DIAPHRAGM ASSEMBLY INCLUDING PULLER WITH RETAINING LEDGE

(71) Applicant: Medela Holding AG, Baar (CH)

(72) Inventors: Joseph Wach, Ingleside, IL (US); Kalpesh Patel, McHenry, IL (US)

(21) Appl. No.: 16/208,817

(22) Filed: Dec. 4, 2018

Related U.S. Application Data
(60) Provisional application No. 62/594,668, filed on Dec. 5, 2017.

(51) Int. Cl. F04B 45/047; A61M 1/00; F04B 43/00; F04B 49/22

(52) U.S. Cl. CPC F04B 45/047; F04B 49/225; F04B 43/0063; A61M 1/0072

(57) ABSTRACT

A diaphragm assembly including a flexible diaphragm and a puller. The diaphragm has a front face and an opposing back face, and a top opening formed through the diaphragm and a bottom opening. The puller has a diaphragm end, a cam end, a puller body extending between the diaphragm end and the cam end along a puller longitudinal axis, and a first retaining ledge extending laterally from the puller body with respect to the puller longitudinal axis. The puller body is disposed through the top opening with the diaphragm disposed between the diaphragm end and the first retaining ledge and with the diaphragm end disposed adjacent the front face and the first retaining ledge disposed adjacent the back face. A method of assembly of the diaphragm assembly is provided.

### CROSS-REFERENCE TO RELATED APPLICATIONS

This application relates to and claims the benefit of and priority to U.S. Provisional Patent Application No. 62/594,668, entitled IMPROVED COMPONENT ASSEMBLY FOR PUMPING SYSTEM, filed Dec. 5, 2017, the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present disclosure relates generally to improved components of a pumping system. More particularly, the disclosure relates to an improved diaphragm assembly for a pumping system.

### 2. Related Art

Certain pumping systems, such as breast pumps and wound therapy pumps, have vacuum generating assemblies within a vacuum chamber of the pumping system. Such systems can include complex connections between components to meet product life requirements, which also add cost to the device. As illustrated in FIGS. 1 and 2, an example of a known assembly for a diaphragm assembly 10 for a pump system can include a diaphragm 12 over-molded upon a puller 14, and a flexible cap 16 supporting back motion of the diaphragm 12 in conjunction with a retaining bar or spring pin 18. The retaining bar or spring pin 18 is disposed through an aperture 20 of the puller 14. Certain components of such a system are illustrated, for example, in FIG. 9 of U.S. Pat. No. 6,997,897, incorporated herein by reference in the entirety. The diaphragm 12 includes a front face 22 and a back face 24. The diaphragm assembly 10 maintains all actuating components for actuating the diaphragm assembly 10, such as the puller 14, and a cam 26 operatively connected thereto on a back face 24 of the diaphragm 12, such that none of the actuating components of the diaphragm assembly 10 are visible along the front face 22 of the diaphragm 12 once assembled. However, costs and manufacturing complexities arise from the over-molding process, and additional components are required to manufacture and assemble such a diaphragm assembly 10.

In view of the foregoing, there is a need in the art for an improved diaphragm assembly in comparison to the prior art.

### BRIEF SUMMARY

In accordance with one embodiment, there is provided a diaphragm assembly. The diaphragm assembly includes a flexible diaphragm and a puller. The flexible diaphragm has a front face and an opposing back face, and a top opening formed through the diaphragm and a bottom opening. The puller has a diaphragm end, a cam end, a puller body extending between the diaphragm end and the cam end along a puller longitudinal axis, and a first retaining ledge extending laterally from the puller body with respect to the puller longitudinal axis. The puller body is disposed through the top opening with the diaphragm disposed between the diaphragm end and the first retaining ledge and with the diaphragm end disposed adjacent the front face and the first retaining ledge disposed adjacent the back face.

According to various embodiments, the diaphragm assembly may further include a cap having a cap central opening concentrically disposed adjacent the top opening at the back face. The puller body is disposed through the cap central opening. The first retaining ledge may include a cap engagement surface facing the diaphragm end. The cap engagement surface may be engaged with the cap with the cap disposed between the cap engagement surface and the back face of the diaphragm. The cap may have a stiffness greater than a stiffness of the diaphragm. The cap may be flexible. The diaphragm may be compressed between the first retaining ledge and the diaphragm end. The puller may be snap-fit through the top opening of the diaphragm with the first retaining ledge engaged with the cap. The diaphragm assembly may further include a cap ring concentrically disposed about the cap with the cap disposed between the cap ring and the first retaining ledge of the puller. The puller may further include a second retaining ledge extending laterally from the puller body and opposite the first retaining ledge with respect to the puller longitudinal axis. The puller may be snap-fit through the diaphragm with the second retaining ledge engaged with the cap. The first retaining ledge may have a tapered surface that is laterally more distant from the puller longitudinal axis in a direction from the cam end toward the diaphragm end. The puller may further include a second retaining ledge extending laterally from the puller body and opposite the first retaining ledge with respect to the puller longitudinal axis. The first retaining ledge and the puller body may be integrally formed of a single contiguous piece of material. The diaphragm assembly may further include a cam rotatably attached to the puller adjacent the cam end.

According to another aspect of the present invention, there is provided a method of assembly of a diaphragm assembly. The method includes providing a flexible diaphragm having a front face and an opposing back face. The flexible diaphragm further has a top opening formed through the diaphragm and a bottom opening. The method further includes providing a puller. The puller has a diaphragm end, a cam end, and a puller body extending between the diaphragm end and the cam end along a puller longitudinal axis. The puller further has a first retaining ledge extending laterally from the puller body with respect to the puller longitudinal axis. The method further includes inserting the cam end through the top opening from the front face of the diaphragm until the first retaining ledge of the puller is disposed at the back face of the diaphragm.

According to various embodiments, the method of assembly may further include compressing the diaphragm between the first retaining ledge and the diaphragm end. The method of assembly may further include providing a cap having a cap central opening concentrically disposed adjacent the top opening at the back face. The inserting of the cam end may further include inserting the cam end through the cap central opening until the first retaining ledge engages the cap with the cap and the diaphragm disposed between the diaphragm end and the first retaining ledge. The method of assembly may further include attaching a cap ring concentrically disposed about the cap with the cap disposed between the cap ring and the first retaining ledge. The cap ring may be over-molded upon the cap.

The present invention will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is a bottom perspective view of a prior art diaphragm assembly;

FIG. 2 is a bottom view of the prior art diaphragm assembly of FIG. 1;

FIG. 3 is a bottom perspective view of the diaphragm assembly according to an aspect of the present invention;

FIG. 4 is a bottom view of the diaphragm assembly of FIG. 3;

FIG. 5 is a left side view of the diaphragm assembly of FIG. 3;

FIG. 6 is a right side view of the diaphragm assembly of FIG. 3;

FIG. 7 is a front view of the diaphragm assembly of FIG. 3;

FIG. 8 is a back view of the diaphragm assembly of FIG. 3;

FIG. 9 is a top view of the diaphragm assembly of FIG. 3;

FIG. 10 is a bottom view the diaphragm of FIG. 9;

FIG. 11 is a cross sectional side view of the diaphragm as depicted along axis 11-11 of FIG. 10;

FIG. 12 is a cross sectional side view of the diaphragm assembly as depicted along axis 12-12 of FIG. 5;

FIG. 13 is an exploded view of the diaphragm assembly of FIG. 12; and

FIG. 14 is a symbolic schematic diagram of the diaphragm assembly with a pump system and a drive mechanism.

Common reference numerals are used throughout the drawings and the detailed description to indicate the same elements.

### DETAILED DESCRIPTION

The detailed description set forth below in connection with the appended drawings is intended as a description of certain embodiments of the present disclosure, and is not intended to represent the only forms that may be developed or utilized. The description sets forth the various functions in connection with the illustrated embodiments, but it is to be understood, however, that the same or equivalent functions may be accomplished by different embodiments that are also intended to be encompassed within the scope of the present disclosure. It is further understood that the use of relational terms such as top and bottom, first and second, and the like are used solely to distinguish one entity from another without necessarily requiring or implying any actual such relationship or order between such entities. Further, the various features of the embodiments disclosed herein can be used alone, or in varying combinations with each other and are not intended to be limited to the specific combination described herein. Thus, the scope of the claims is not to be limited by the illustrated embodiments.

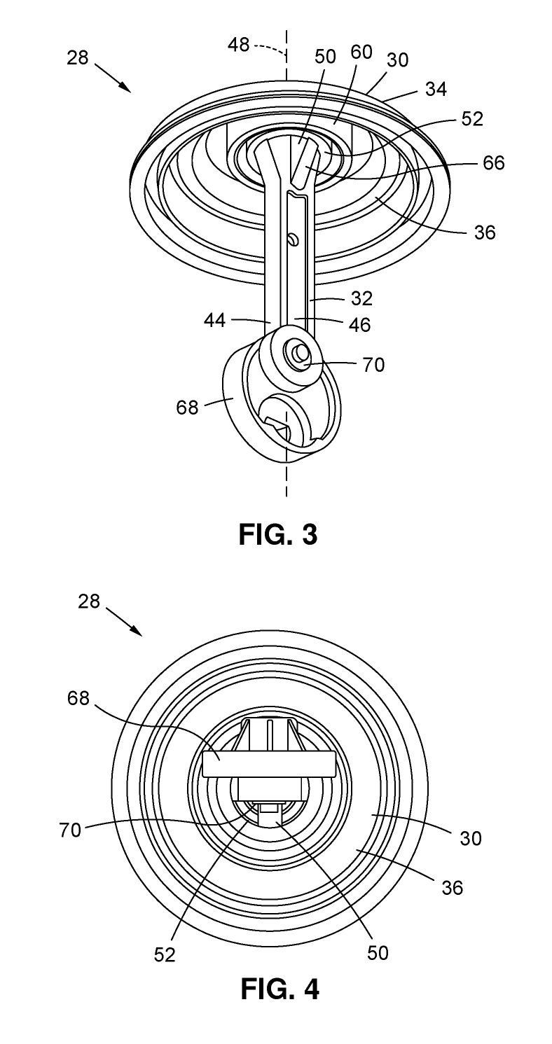

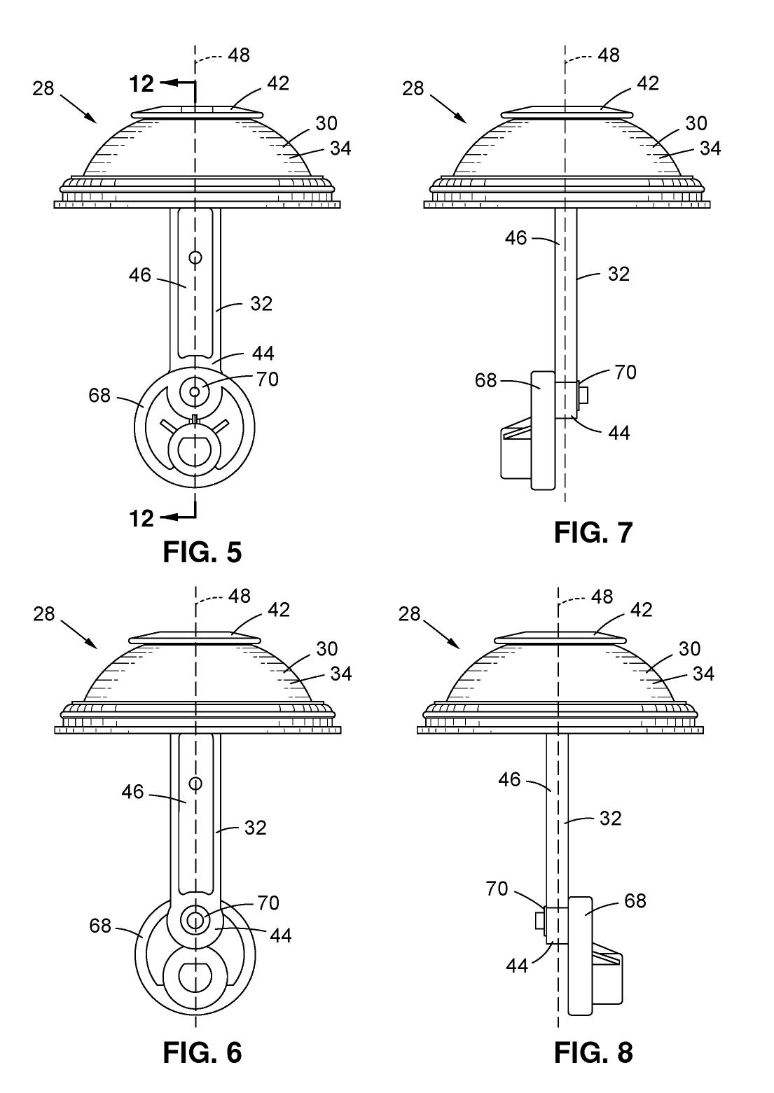

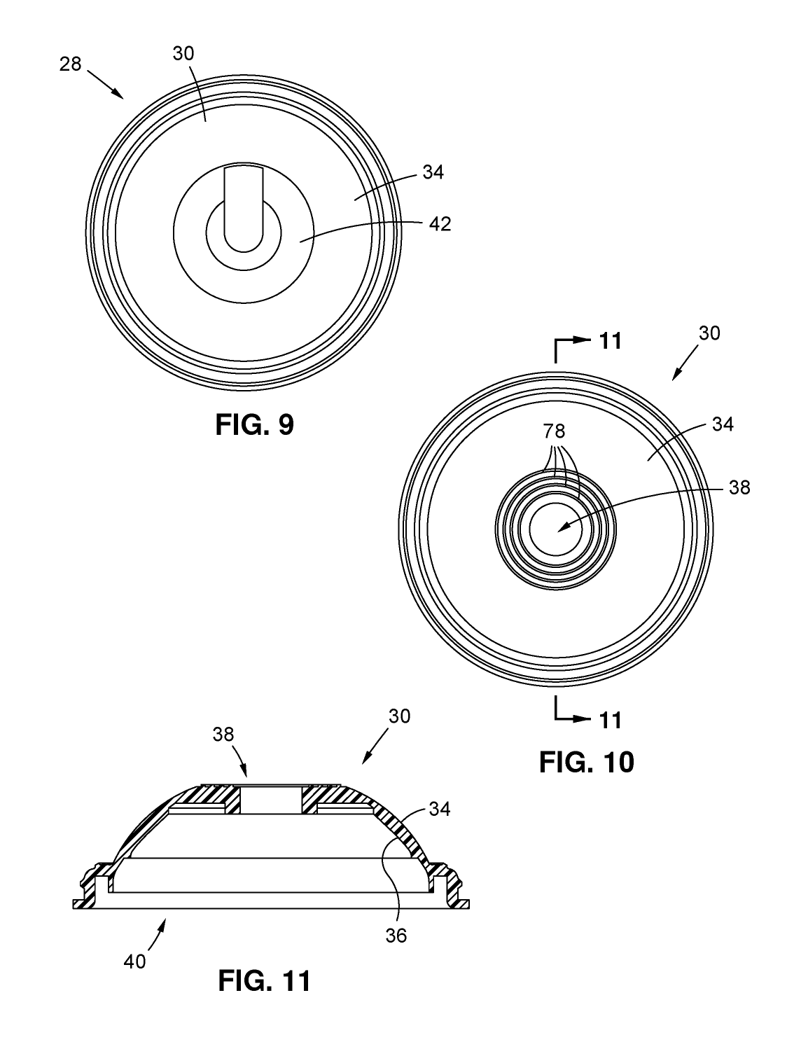

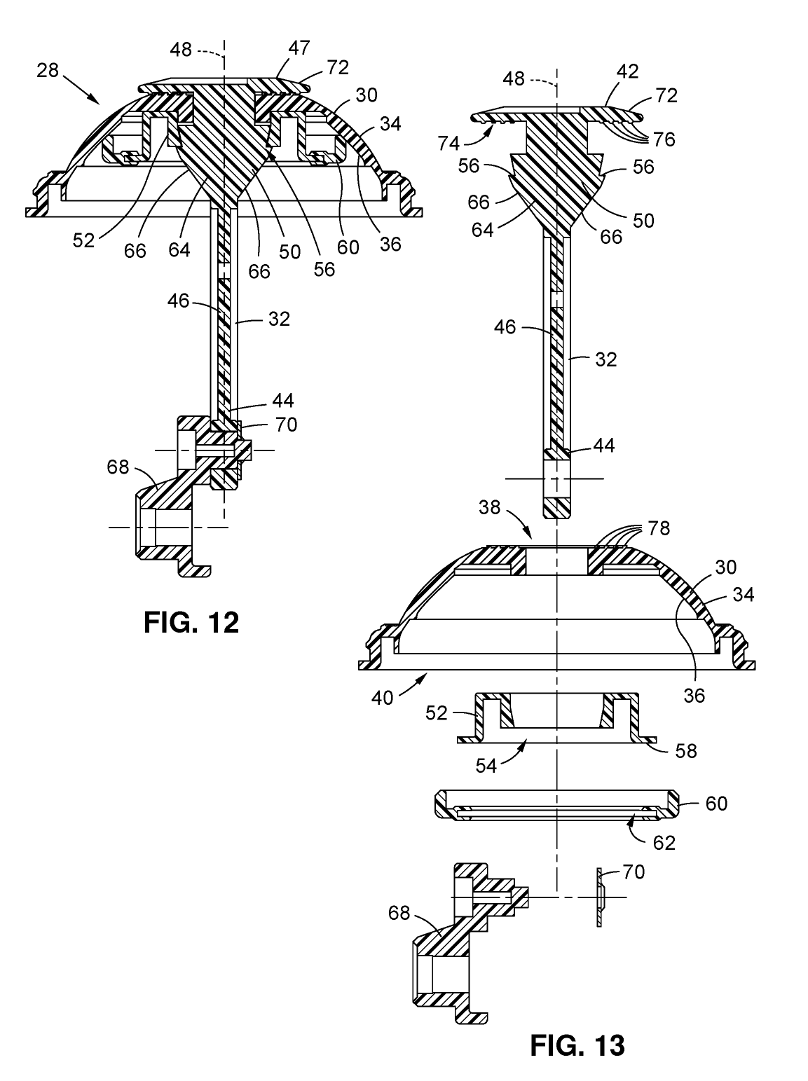

Referring now to FIG. 3, there is depicted a bottom perspective view of a diaphragm assembly 28 according to an aspect of the present invention. FIGS. 4-8 are respectively bottom, left side, right side, front and back views of the diaphragm assembly 28. FIGS. 9 and 10 are respectively top and bottom views of a diaphragm 30 of the diaphragm assembly 28. FIG. 11 is a cross sectional side view of the diaphragm 30 as depicted along axis 11-11 of FIG. 10, and FIG. 12 is a cross sectional side view of the diaphragm assembly 28 as depicted along axis 12-12 of FIG. 5. FIG. 13 is an exploded view of the diaphragm assembly 28 of FIG. 12.

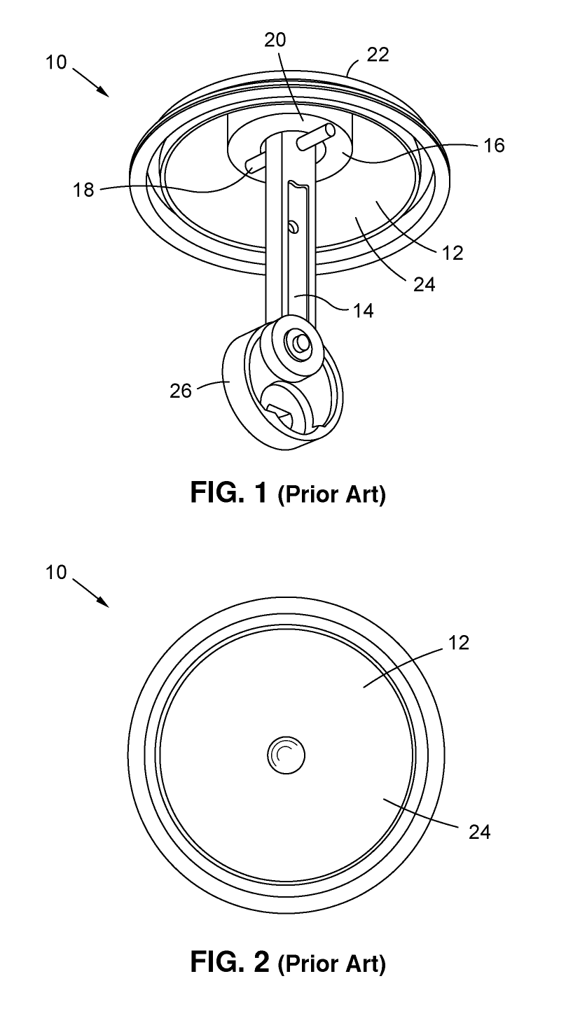

According to an embodiment of the invention, there is provided the diaphragm assembly 28. The diaphragm assembly 28 includes the flexible diaphragm 30 and a puller 32. The flexible diaphragm 30 has a front face 34 and an opposing back face 36, and a top opening 38 formed through the diaphragm 30 and a bottom opening 40. The puller 32 has a diaphragm end 42, a cam end 44, and a puller body 46 extending between the diaphragm end 42 and the cam end 44 along a puller longitudinal axis 48. Importantly, the puller 32 includes a first retaining ledge 50 extending laterally from the puller body 46 with respect to the puller longitudinal axis 48. The puller body 46 is disposed through the top opening 38 of the diaphragm 30 with the diaphragm 30 disposed between the diaphragm end 42 and the first retaining ledge 50 and with the diaphragm end 42 disposed adjacent the front face 34 of the diaphragm 30 and the first retaining ledge 50 disposed adjacent the back face 36 of the diaphragm 30. As will be discussed further below, the foregoing diaphragm assembly 28 facilitates an improved design that may eliminate or mitigate costs and complexities over prior art designs, such as depicted in FIGS. 1 and 2 and reduces the number of components required to form a suitable diaphragm assembly.

According to various embodiments, the diaphragm assembly 28 may further include a cap 52 having a cap central opening 54 (as best viewed in FIG. 13) concentrically disposed adjacent the top opening 38 at the back face 36. The puller body 46 is disposed through the cap central opening 54. The first retaining ledge 50 may include a cap engagement surface 58 (as best viewed in FIG. 13) facing the diaphragm end 42. The cap engagement surface 56 of the puller 32 may be engaged with the cap 52, with the cap 52 disposed between the cap engagement surface 56 and the back face 36 of the diaphragm 30. The cap 52 may have a stiffness greater than a stiffness of the diaphragm 30, although the cap 52 may be somewhat flexible. The puller 32 may be snap-fit through the top opening 38 and the cap 52 with the first retaining ledge 50 engaged with the cap 52. With the foregoing configuration, the diaphragm 30 may be compressed between the first retaining ledge 50 and the diaphragm end 42. Such compression is used to provide a vacuum seal between the cap end 44 of the puller 32 and the diaphragm 30. In this regard, an aspect of the present invention is that the puller 32 may be attached to the diaphragm 30 in sealed engagement while avoiding the diaphragm 30 having to be over-molded upon any other components, such as a cap or the like, and thereby avoiding the costs and complexities associated therewith.

The cap 52 may further include an outward facing flange 58. The outward facing flange 58 is generally radially disposed and includes a circular edge. The diaphragm assembly 28 may further include a cap ring 60. The cap ring 60 includes an inward facing circular slot 62. The cap ring 60 may be attached to the cap 52 with the outward facing flange 58 disposed in the inward facing slot 62. The cap ring 60 is concentrically disposed about the cap 52 with the cap 52 disposed between the cap ring 60 and the first retaining ledge 50 of the puller 32. The cap ring 60 may be over-molded upon the cap 52 and may be formed of a material having a stiffness less than the stiffness of the cap 52. In this regard, the cap ring 60 may be more flexible and softer than the cap 52. The cap ring 60 may be disposed in contact with the back face 36 of the diaphragm 30.

As the cap ring 60 may be configured to contact the back face 36 of the diaphragm 30. Such contact may be used to push out the diaphragm 30 from the back face 36 in a direction of the front face 34 so as to spread the loading upon the diaphragm 30 and lessen wear at other locations about the diaphragm 30 (such as immediately adjacent the contact locations of the diaphragm end 42 of the puller 32 and the first retaining ledge 50). The contact by the cap ring 60 may also provide a degree of sealing with the diaphragm 30. The cap ring 60 may also provide a degree of structural to support to the cap 52 to maintain the structural shape to the cap 52 as the cap 52 is exposed to compression forces with the diaphragm 30 being compressed in engagement with diaphragm end 42 of the puller 32.

The puller 32 may further include a second retaining ledge 64 extending laterally from the puller body 46 and opposite the first retaining ledge 50 with respect to the puller longitudinal axis 48. The puller 32 may be snap-fit through the diaphragm 30 with the second retaining ledge 64 also engaged with the cap 52. In this regard, it is contemplated that there may be any number of addition retaining ledges similar to or different from the first and second retaining ledges 50, 64.

The first retaining ledge may have a tapered surface 66 that is laterally more distant from the puller longitudinal axis in a direction from the cam end 44 toward the diaphragm end 42. Such tapered surface 66 is contemplated to facilitate slidable contact between the first retaining ledge 50 and the diaphragm 50 at the top opening 38 upon insertion of the puller 32 and passage of the first retaining ledge 50 through the top opening 38. However, rather than a smooth tapered surface 66, a more stepped surface may be used.

It is contemplated that other configurations of the first and second retaining ledges 50, 64 may be implemented. In this regard, as used herein the terms first and second retaining ledges 50, 64 refer to a structure that laterally extends or protrudes from the puller body 46 capable of engagement or interference with a counter-force acting in a direction opposite to a movement of the puller 30 in a direction from the cam end 44 towards the diaphragm end 42 along the puller longitudinal axis 48. Such configuration would also need to consider the degree of elastomeric deformation of the diaphragm 30 at the top opening 38 that may be required when inserting the puller 32 there through, and in particular the passage of the first and second retaining ledges 50, 64 there through.

The first and second retaining ledges and the puller body may be integrally formed of a single contiguous piece of material. In this regard, the first and second ledges 50, 64 may be used to engage the puller 32 with the diaphragm 30 in sealed engagement without the need for an additional separate attachment component, like a retaining bar, a spring pin or other fastener. This eases the task of assembly and reduces the overall number of components of the overall diaphragm assembly 28.

It is contemplated that the materials and techniques used to manufacture of the various components of the diaphragm assembly 28 (such as the diaphragm 30, the puller 32, the cap 52 and the cap ring 60) may be chosen from those which are well known to one of ordinary skill in the art and may include, for example, injection molding and over-molding processes with various types of plastics and elastomeric materials as may be suitable for the particular intended applications.

The diaphragm end 42 of the puller 32 may include an end flange 72 with an overhang annular surface 74 facing the cam end 44. The overhang annular surface 74 may be disposed in contact with the diaphragm 30 about the top opening 38 at the front face 34. The overhang annular surface 74 may include concentric ridges 76. In this regard the diaphragm 30 may include annular ribs 78 which extend from the front face 34 about the top opening 38. The concentric ridges 76 may be cooperatively sized and configured with the annular ribs 78 and interspersed alternatively to form a sealing engagement between the diaphragm end 42 of the puller 32 and the diaphragm 30 at the top opening 38. The annular ribs 78 may be relatively thin with a short profile so as to be crushable or readily deformable upon exposure to compressive forces. As such, a vacuum seal may be achieved with respect to the interior of the diaphragm 30 so as to prevent any gas passage between adjacent the front face 34 and the back face 36 of the diaphragm 30 through the top opening 38.

The diaphragm assembly 28 may further include a cam 68 rotatably attached to the puller 32 adjacent the cam end 44. It is understood that the puller 32, as the name suggests, is used to repetitively pull the diaphragm 30. Such reciprocating movement may be achieved through the rotation of the cam 68. The cam 68 may be constructed and attached to the puller 32 in accordance to any of those techniques and materials which are well known to one of ordinary skill in the art.

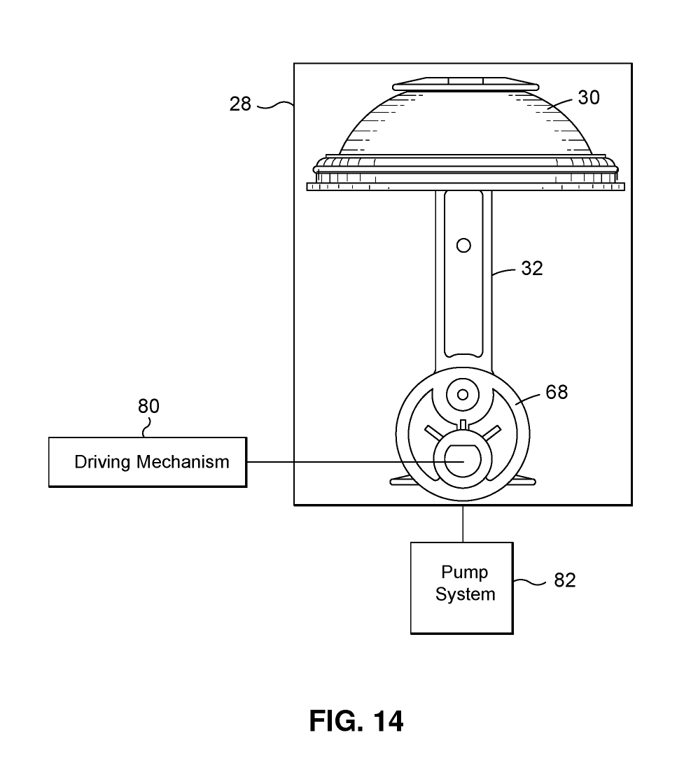

With reference to the symbolic schematic diagram of FIG. 14, it is understood that diaphragm assembly 26 is suitable for use with a pump system 82. In this regard, it is further understood that the diaphragm 30 may be used to form a portion of a vacuum chamber. In addition there is provided a drive mechanism 80 for powering reciprocating movement of the puller 32.

According to another aspect of the present invention, there is provided a method of assembly of the diaphragm assembly 28. The method includes providing the flexible diaphragm 30 having the front face 34 and the opposing back face 36, and the top opening 38 formed through the diaphragm 30 and the bottom opening 40. The method further includes providing the puller 30 having the diaphragm end 42, the cam end 44, the puller body 46 extending between the diaphragm end 42 and the cam end 44 along the puller longitudinal axis 48, and the first retaining ledge 50 extending laterally from the puller body 46 with respect to the puller longitudinal axis 48. The method further includes inserting the cam end 44 through the top opening 38 from the front face 34 until the first retaining ledge 50 is disposed at the back face 36.

According to various embodiments, the method of assembly of the diaphragm assembly 28 may further include compressing the diaphragm 30 between the first retaining ledge 50 and the diaphragm end 42 of the puller 32. The method of assembly of the diaphragm assembly 28 may further include providing the cap 52 having the cap central opening 54 concentrically disposed adjacent the top opening 38 at the back face 36 of the diaphragm 30. The inserting of the cam end 44 may further include inserting the cam end 44 through the cap central opening 54 until the first retaining ledge 50 engages the cap 52, with the cap 52 and the diaphragm 30 disposed between the diaphragm end 42 and the first retaining ledge 50 of the puller 32. It is understood that the insertion of the cam end 44 refers to relative movement of the puller 32 to the diaphragm 30 and the cap 52. As such, such insertion of the cam end 44 may include movement of the cap 52 about the cam end 44 and along the puller body 46 while the puller 52 is stationary.

The method of assembly of the diaphragm assembly 28 may further include attaching the cap ring 60 concentrically disposed about the cap 52. The cap 52 is disposed between the cap ring 60 and the first retaining ledge 50 of the puller 32. The cap ring 60 may be over-molded upon the cap 52. In this regard, the cap 52 as combined with the cap ring 60 may be attached to the puller 32.

The particulars shown herein are by way of example only for purposes of illustrative discussion, and are presented in the cause of providing what is believed to be the most useful and readily understood description of the principles and conceptual aspects of the various embodiments set forth in the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A diaphragm assembly comprising:
a flexible diaphragm having a front face and an opposing back face, and a top opening formed through the diaphragm and a bottom opening; and
a puller having a diaphragm end, a cam end, a puller body extending between the diaphragm end and the cam end along a puller longitudinal axis, and a first retaining ledge extending laterally from the puller body with respect to the puller longitudinal axis, the puller body being disposed through the top opening with the diaphragm disposed between the diaphragm end and the first retaining ledge and with the diaphragm end disposed adjacent the front face and the first retaining ledge disposed adjacent the back face.

2. The diaphragm assembly of claim 1 further includes a cap having a cap central opening concentrically disposed adjacent the top opening at the back face, the puller body is disposed through the cap central opening

3. The diaphragm assembly of claim 2 wherein the first retaining ledge includes a cap engagement surface facing the diaphragm end, the cap engagement surface is engaged with the cap with the cap disposed between the cap engagement surface and the back face.

4. The diaphragm assembly of claim 2 wherein the cap has a stiffness greater than a stiffness of the diaphragm.

5. The diaphragm assembly of claim 2 wherein the cap is flexible.

6. The diaphragm assembly of claim 2 wherein the puller is snap-fit through the top opening with the first retaining ledge engaged with the cap.

7. The diaphragm assembly of claim 6 further includes a cap ring concentrically disposed about the cap with the cap disposed between the cap ring and the first retaining ledge.

8. The diaphragm assembly of claim 2 wherein the puller further includes a second retaining ledge extending laterally from the puller body and opposite the first retaining ledge with respect to the puller longitudinal axis.

9. The diaphragm assembly of claim 8 wherein the puller is snap-fit through the diaphragm with the second retaining ledge engaged with the cap.

10. The diaphragm assembly of claim 8 wherein the diaphragm is compressed between the first retaining ledge and the diaphragm end.

11. The diaphragm assembly of claim 1 wherein the first retaining ledge has a tapered surface that is laterally more distant from the puller longitudinal axis in a direction from the cam end toward the diaphragm end.

12. The diaphragm assembly of claim 1 wherein the puller further includes a second retaining ledge extending laterally from the puller body and opposite the first retaining ledge with respect to the puller longitudinal axis.

13. The diaphragm assembly of claim 1 wherein the first retaining ledge and the puller body are integrally formed of a single contiguous piece of material.

14. The diaphragm assembly of claim 1 further includes a cam rotatably attached to the puller adjacent the cam end.

15. A method of assembly of a diaphragm assembly, the method comprising:
providing a flexible diaphragm having a front face and an opposing back face, and a top opening formed through the diaphragm and a bottom opening; and
providing a puller having a diaphragm end, a cam end, a puller body extending between the diaphragm end and the cam end along a puller longitudinal axis, and a first retaining ledge extending laterally from the puller body with respect to the puller longitudinal axis, and
inserting the cam end through the top opening from the front face until the first retaining ledge is disposed at the back face.

16. The method of assembly of claim 15 further includes:
compressing the diaphragm between the first retaining ledge and the diaphragm end.

17. The method of assembly of claim 15 further includes:
providing a cap having a cap central opening concentrically disposed adjacent the top opening at the back face.

18. The method of claim 17 wherein the inserting the cam end further includes inserting the cam end through the cap central opening until the first retaining ledge engages the cap with the cap and the diaphragm disposed between the diaphragm end and the first retaining ledge.

19. The method of claim 18 further includes:
attaching a cap ring concentrically disposed about the cap with the cap disposed between the cap ring and the first retaining ledge.

20. The method of claim 18 wherein the cap ring is over-molded upon the cap.
